(19) United States
(12) Patent Application Publication
(10) Pub. No.: US 2025/0043230 A1
(43) Pub. Date: Feb. 6, 2025

(54) SELECTIVE SEPARATION OF AMMONIUM AND LACTATE FROM CELL CULTURE MEDIA

(71) Applicant: B. G. NEGEV TECHNOLOGIES AND APPLICATIONS LTD., AT BEN-GURION UNIVERSITY, Beer-Sheva (IL)

(72) Inventor: Oded NIR, Midreshet Ben Gurion (IL)

(21) Appl. No.: 18/718,176

(22) PCT Filed: Dec. 28, 2022
(86) PCT No.: PCT/IL2022/051406

§ 371 Date: Jun. 10, 2024

Related U.S. Application Data
(60) Provisional application No. 63/398,376, filed on Aug. 16, 2022.
(60) Provisional application No. 63/294,432, filed on Dec. 29, 2021.

(51) Int. Cl. C12M 1/34; C12M 1/00; C12M 1/36

(52) U.S. Cl. CPC C12M 41/32; C12M 29/04; C12M 29/06; C12M 41/48

(57) ABSTRACT

There are provided herein systems and methods for recycling culture media by removing undesired materials, such as, ammonium and lactate therefrom, which are safe, efficient, cost effective, and able to specifically remove selected undesired materials from culture media, while maintaining vital material, such as growth factors, an amino acids, vitamins, proteins, enzymes, co-enzymes, hormones, sugars, carbohydrates, micronutrients, macronutrients, minerals, osmolarity agents, pH maintenance agents, under various conditions and settings.

### TECHNICAL FIELD

The present disclosure relates generally to systems and methods for recycling culture media by removing undesired material therefrom.

### BACKGROUND

The growth of cell tissues (cell multiplication) in a culture media form the basis of many production processes in the biotechnology field. Some specific examples include cultivated meat production, cultivated plant production, enzyme production, alternative protein production, growth of artificial organs, metabolites, and production of pharmaceutically active components.

The cultivation of various tissues (plant, animal, and microorganism) is becoming increasingly important in the fields of food technology, pharmaceuticals, and cosmetics. For example, the cultivation of animal cells for the production of synthetic meats, vaccines, biological medication, hormones, cancer research, etc. Additionally, there is extensive work in the cultivation of plant cells, particularly for plant products that are rare, expensive, or difficult to grow or collect (e.g., cocoa, vanilla, saffron, coffee, truffle, black or green cardamom, kaffir lime, wasabi, fennel pollen, etc.). Further, yeast is used in bioreactors for the production of many substances, such as those produced by various enzymatic or metabolic pathways, which may be genetically engineered.

Among the challenges of bringing up the biological process to an industrial scale, liquid waste management and recycling water and nutrients are major bottlenecks. On a small scale, wastewater production and water usage are economically insignificant; and may be addressed using techniques that are not optimal in terms of energy consumption, footprint, cost, and environmental protection. However, on expanding to an industrial scale, the most cost-efficient and eco-efficient technologies must be used to enable economic and sustainable cellular agriculture.

The culture or growth medium is an aqueous environment in which the target cells multiplicate. It contains all the vital materials such as macronutrients and micronutrients required for cell growth and organic growth factors such as hormones, enzymes, and co-enzymes. In addition, it includes an adequate amount of salt, commonly sodium chloride, to maintain the desired osmolarity and a weak-acid buffer, commonly phosphate-based, to maintain the desired pH. It is essential to sustain a constant composition in the culture medium for optimal growth.

However, as part of the metabolic processes associated with cell growth, waste products such as, metabolites, alcohol, alkaloids, carbon dioxide, lactate and ammonium ions are generated, and accumulated in the culture media. These components can significantly inhibit cell growth even at low concentrations (several millimolar). Therefore, to achieve cell multiplication at reasonable rates, the spent culture media must be replaced by fresh culture media frequently, losing all other precious beneficial components. This practice, currently applied in small-scale reactors, is unsuitable for large-scale operations due to the high cost of the media culture. Accumulation of undesired materials therefore remains a primary roadblock and a bottleneck for the large-scale application of cell-growth reactors.

Selective and specific separation of undesired materials such as ammonium and lactate from culture media could facilitate the scaling-up of cell-growth bioreactors. However, a cost-effective method is currently unavailable, neither in the market nor in professional literature. Developing such a method is challenging since ultra-selectivity for the undesired materials is required without altering the composition of the culture medium. Separation processes that induce a change in temperature, pH, salt concentration, and organic matter composition cannot be used.

Technologies from the field of water and wastewater treatment operate on very large scales (e.g., desalination plants and wastewater treatment plants) and are highly optimized for low energy consumption, low footprint, and low overall cost. Therefore, cutting-edge water treatment technologies such as electrodialysis, ion exchange, adsorption, membrane filtration, and others are more suitable on scale-up than standard bio-separation processes, such as, chromatography and centrifugation. However, a high level of innovation is required to adjust water treatment technologies to the specific needs of the cell and tissue culture industry. Such innovation must draw from a comprehensive knowledge of different technologies and a deep understanding of the mechanisms driving selective solute separations.

In contrast to conventional water and wastewater treatment which remove most compounds and particles from the aqueous media, recycling culture media requires selectively maintaining and/or recycling vital materials while removing waste products. Such ultra-selectivity is challenging, particularly in culture media which comprise a wide variety of macronutrients and micronutrients where the variation between the compounds is high (e.g., various sizes, polarities, charges, acidity, etc.). Moreover, the complexity is increased since the system must be closed, and sterile conditions must be maintained to prevent growth of unwanted microorganisms. Additionally, changes in temperature, pH, salt concentration, and organic matter composition must be avoided.

Selective adsorption and ion exchange has been investigated as a possible solution. However, the selectivity of the sorbents was only shown in simple mixtures containing glucose and not in actual culture media, which includes various components. Also, one-module electrodialysis was previously suggested, but no selective removal of undesired materials over salt was reported. Further, recovering both organic and inorganic compounds from spent culture media would significantly improve the cost-effectiveness and reduce the environmental impact of the production process.

There is thus a need in the art for a selective process for the removal of undesired materials such as lactate and ammonium from spent growth media, which is effective, selective, cost effective, while maintaining the vital materials in the culture media.

### SUMMARY

According to some embodiments, provided herein are systems and methods for recycling culture media by removing undesired material therefrom, which are safe, efficient, cost effective, and able to specifically remove selected undesired materials from culture media, under various conditions and settings.

According to some embodiments, the recycling system for removing at least one undesired material from spent culture media, the system may include:
at least one culture media reservoir;
at least one electrolyte solution reservoir;
at least one electrodialysis module; and
at least one ion exchange module;
wherein the system may be configured to remove an electrolyte and at least one undesired material from the spent culture media, thereby producing an undesired material reduced culture media, utilizing the at least one electrodialysis module and the at least one ion exchange module, wherein the system may be further configured to recover the removed electrolyte, and optionally, also any vital materials that were removed with the electrolyte, and to return the electrolyte and/or any removed vital materials to the undesired material reduced culture media, which may then be returned to the at least one culture media reservoir.

According to some embodiments, the electrolyte may be a salt solution including sodium, calcium, potassium, chloride, phosphate, carbonate, bicarbonate, magnesium, sulfonic acid, sulfate, nitrate, organic salts like acetate, citrate, formate, a derivative thereof or any combination thereof. Each possibility is a separate embodiment. According to some embodiments, the electrolyte may include a NaCl solution.

According to some embodiments, the undesired material may be a growth inhibitor, waste product, impurity, contaminant, metabolite, excess of one or more culture media components, or a combination thereof. Optionally, the undesired material may be selected from a group consisting of ammonium, lactate, lactose, hydrogen, alcohol (e.g., ethanol), alkaloid, carbon dioxide, uric acid, urea, creatine, creatinine, an amino acid or any combination thereof. Each possibility is a separate embodiment. Optionally, the growth inhibitor may be selected from a group consisting of ammonium, lactate, or both. Each possibility is a separate embodiment.

According to some embodiments, the system may be configured to return to the undesired material reduced culture media, a vital material that was removed during the recycling process. According to some embodiments, the system may be configured to replace a depleted vital material in the culture media.

According to some embodiments, the vital material may be a growth factor, an amino acid, a vitamin, a protein, an enzyme, a co-enzyme, a hormone, a sugar, a carbohydrate, a micronutrient, macronutrient, a mineral, an osmolarity agent, a pH maintenance agent, or any combination thereof. Each possibility is a separate embodiment.

According to some embodiments, the order of the at least one electrodialysis module and the at least one ion exchange module may be variable.

According to some embodiments, the culture media may be used for cultivated meat production, cultivated plant production, alternative protein production, enzyme production, growth of artificial organs, metabolites, production of pharmaceutically active components, or any combination thereof. Each possibility is a separate embodiment. Optionally, the culture media may be used to cultivate tissue including animal tissue, plant tissue, fungal, algal tissue, or any combination thereof. Each possibility is a separate embodiment. Optionally, the culture media may be used to cultivate cells including animal cells, plant cells, bacteria, yeast, fungi, microalgae, algae or any combination thereof. Each possibility is a separate embodiment.

According to some embodiments, the culture media may include a growth factor, an amino acid, a vitamin, a protein, an enzyme, a co-enzyme, a hormone, a sugar, a carbohydrate, a micronutrient, macronutrient, a mineral, an osmolarity agent, a pH maintenance agent, and combinations thereof.

According to some embodiments, the recycling system may be configured to repeat the modules of growth inhibitor removal and/or salt recovery n times, wherein n>1. According to some embodiments, the recycling system may be configured to operate as a continuous process, semi-batch process, and/or a batch process.

According to some embodiments, the ion exchange module may include an ion exchanger selected from a membrane, a column, a bed, or suspended beads. According to some embodiments, the ion exchange module may include an ion selective ion exchanger. Optionally, the ion exchanger may be selective for at least one undesired material.

According to some embodiments, the at least one electrodialysis module may include a membrane selective for an undesired material.

According to some embodiments, the recycling system for removing a growth inhibitor from spent culture media, the system may include:
a culture media reservoir;
an electrolyte solution reservoir;
a first electrodialysis module configured to receive:
   spent culture media from the culture media reservoir as a first diluate; and
   an electrolyte solution from the electrolyte solution reservoir as a first concentrate,
wherein the first electrodialysis module may be configured to remove ammonium from the received spent culture media and to output a reduced ammonium culture media and/or an ammonium containing concentrate;
a second electrodialysis module configured to receive:
   the reduced ammonium culture media as a second diluate; and
   an electrolyte solution, from the electrolyte solution reservoir as a second concentrate,
wherein the second electrodialysis may be configured to remove lactate from the reduced ammonium culture media and to output a reduced ammonium and lactate culture media and/or a lactate containing solution;
an ion exchange module which may be configured to receive the ammonium containing concentrate, to remove the ammonium and to output a reduced ammonium solution; and
a third electrodialysis module configured to receive:
   the reduced ammonium solution as diluate; and
   the reduced ammonium and lactate culture media as a third concentrate,
wherein the third electrodialysis module may be configured to return the reduced ammonium solution to the reduced ammonium and lactate culture media; and to output:
   an electrolyte enriched, ammonium reduced and lactate reduced culture media to be returned to the culture media reservoir thereby purifying the spent culture media, and optionally
   an electrolyte solution to be returned to the electrolyte solution reservoir for re-use as concentrate for the first dialysis module and/or second dialysis module.

According to some embodiments, the recycling system for removing ammonium and lactate from spent culture media, the system may include:
a culture media reservoir;
an electrolyte solution reservoir;
a first electrodialysis module configured to receive:
   spent culture media from the culture media reservoir as a first diluate; and
   an electrolyte solution from the electrolyte solution reservoir as a first concentrate,
wherein the first electrodialysis module may be configured to remove ammonium from the received spent culture media and to output a reduced ammonium culture media and/or an ammonium containing concentrate;
a second electrodialysis module configured to receive:
   the reduced ammonium culture media as a second diluate; and
   an electrolyte solution from the electrolyte solution reservoir as a second concentrate,
wherein the second electrodialysis may be configured to remove lactate from the reduced ammonium culture media and to output a reduced ammonium and lactate culture media and/or a lactate containing solution;
an ion exchange module which may be configured to receive the ammonium containing concentrate, to remove the ammonium and to output a reduced ammonium solution which may optionally be returned to the electrolyte solution reservoir for re-use as concentrate for the first dialysis module and/or second dialysis module;
an electrolyte, and/or optionally one or more vital materials, may be added to the reduced ammonium and lactate culture media to make up for the electrolyte, and/or optionally one or more vital materials lost in the first electrodialysis module, and to output an electrolyte enriched ammonium reduced and lactate reduced culture media to be returned to the culture media reservoir thereby purifying the spent culture media.

According to some embodiments, the recycling system for removing a growth inhibitor from spent culture media, the system may include:
a culture media reservoir;
an electrolyte solution reservoir;
a first electrodialysis module configured to receive:
   spent culture media from the culture media reservoir as a first diluate; and
   an electrolyte solution from the electrolyte solution reservoir as a first concentrate,
wherein the first electrodialysis module may be configured to remove ammonium from the received spent culture media and to output a reduced ammonium culture media and/or an ammonium containing concentrate;
a first ion exchange module which may be configured to receive the reduced ammonium culture media, to remove lactate and to output a reduced ammonium and lactate culture media;
a second ion exchange module which may be configured to receive the ammonium containing concentrate, to remove the ammonium and to output a reduced ammonium solution; and
a second electrodialysis module configured to receive:
   the reduced ammonium solution as diluate; and
   the reduced ammonium and lactate culture media as a third concentrate, wherein the second electrodialysis module may be configured to return the reduced ammonium solution to the reduced ammonium and lactate culture media; and
to output:
   an electrolyte enriched ammonium reduced and lactate reduced culture media to be returned to the culture media reservoir thereby purifying the spent culture media; and optionally,
   an electrolyte solution to be returned to the electrolyte solution reservoir for re-use as concentrate for the first dialysis module.

According to some embodiments, the reduced ammonium solution output by the ion exchange module may include one or more vital materials that may be returned to the culture media reservoir through the third electrodialysis module.

According to some embodiments, the amount of electrolyte added to the reduced ammonium and lactate culture media may be up to about 10% v/v.

According to some embodiments, the electrolyte may be a salt solution including sodium, calcium, potassium, chloride, phosphate, carbonate, bicarbonate, magnesium, sulfonic acid, sulfate, nitrate, organic salts like acetate, citrate, formate, a derivative thereof or any combination thereof. Each possibility is a separate embodiment. Optionally, the electrolyte comprises a NaCl solution.

According to some embodiments, the terms “electrolyte” and “salt” may be used interchangeably.

According to some embodiments, the first electrodialysis module, the second electrodialysis module or the third electrodialysis module may include at least one 2-cell repeating unit including an Anion Exchange Membrane (AEM) and a Cation Exchange Membrane (CEM). Optionally, the number of repeat units is in the range between about 1-300 repeating units.

According to some embodiments, wherein the AEM membrane and/or the CEM surface area may be in the range between about 2 cm² up to 2 m² for one repeating unit. Optionally, spacers may be located between the CEM and AEM.

According to some embodiments, the AEM of the first electrodialysis module may be configured to allow passage of small negatively charged ions. According to some embodiments, the CEM of the first electrodialysis module may be configured to be monovalent cation selective. According to some embodiments, the CEM of the first electrodialysis module may be configured to allow passage of cations smaller than about 200 Da.

According to some embodiments, the AEM of the second electrodialysis module may be configured to be selective to lactate. According to some embodiments, the CEM of the second electrodialysis module may be configured to allow passage of small positively charged ions.

According to some embodiments, the AEM of the third electrodialysis module may be configured to be selective, partially selective or non-selective. According to some embodiments, the CEM of the third electrodialysis module may be configured to be selective, partially selective or non-selective.

According to some embodiments, the hydraulic residence time of the first electrodialysis module, second electrodialysis module, and/or third electrodialysis module may be in the range between about 30 seconds to 6 hrs.

According to some embodiments, the current density of the first electrodialysis module, second electrodialysis module, and/or third electrodialysis module may be in the range between about 0.1-2000 A/m².

According to some embodiments, about 75-90% of the ammonium ions may migrate from the diluate to the concentrate of the first electrodialysis module to output the reduced ammonium culture media.

According to some embodiments, up to about 90%, up to about 95% or up to about 99% of the lactate may be removed from the reduced ammonium culture media in the second electrodialysis module to output the reduced ammonium and lactate culture media. Each possibility is a separate embodiment. According to some embodiments, the output lactate containing solution may be further concentrated for disposal and/or reuse.

According to some embodiments, the ion exchange module may include an ion exchanger with high selectivity to ammonium or lactate. According to some embodiments, the ion exchange module may include a cation exchange resin with a high affinity towards ammonium. According to some embodiments, the ion exchange module may include an anion exchange resin with a high affinity towards lactate.

According to some embodiments, the ion exchange module includes a zeolite, copper-based resin polysulphone based, polymer-based, or zinc hexacyanoferrate as the ion exchanger. Each possibility is a separate embodiment.

According to some embodiments, the ion exchange module may be operated as a column, suspension and separation, and/or mixed matrix filter.

According to some embodiments, up to about 90%, up to about 95% or up to about 99% of the ammonium may be removed from the ammonium containing concentrate in an ion exchange module to output the reduced ammonium solution. Each possibility is a separate embodiment. According to some embodiments, up to about 90%, up to about 95% or up to about 99% of the lactate may be removed from the reduced ammonium culture media in an ion exchange module to output the reduced ammonium and lactate culture media. Each possibility is a separate embodiment. According to some embodiments, the output lactate containing solution may be further concentrated for disposal and/or reuse.

According to some embodiments, the electrolyte concentration in the electrolyte enriched reduced ammonium and lactate culture media may be controlled by adjusting current density, voltage, and/or hydraulic residence time, or any combination thereof. Each possibility is a separate embodiment.

According to some embodiments, the method for recycling spent culture media by removing ammonium and lactate may include:
receiving:
spent culture media as a first diluate, from a culture media reservoir; and
an electrolyte solution, from an electrolyte solution reservoir, as a first concentrate;
to a first electrodialysis module configured for removing ammonium from the received spent culture media and outputting a reduced ammonium culture media and an ammonium containing concentrate;
receiving:
the reduced ammonium culture media as a second diluate; and
an electrolyte solution, from an electrolyte solution reservoir, as a second concentrate;
to a second electrodialysis module configured for removing lactate from the reduced ammonium culture media and outputting a reduced ammonium and lactate culture media and a lactate containing solution;
receiving the ammonium containing concentrate to an ion exchange module configured for removing the ammonium and for outputting a reduced ammonium solution;
receiving:
the reduced ammonium solution as diluate and the reduced ammonium and lactate culture media as a third concentrate
to a third electrodialysis module configured for returning the reduced ammonium solution to the reduced ammonium and lactate culture media and for outputting an electrolyte enriched ammonium reduced and lactate reduced culture media to be returned to the culture media reservoir thereby purifying the spent culture media, and
returning an electrolyte solution to the electrolyte solution reservoir for re-using as concentrate for the first dialysis module and second dialysis module.

According to some embodiments, the method for recycling spent culture media by removing ammonium and lactate may include:
receiving:
spent culture media as a first diluate, from a culture media reservoir; and
an electrolyte solution, from an electrolyte solution reservoir, as a first concentrate;
to a first electrodialysis module configured for removing ammonium from the received spent culture media and outputting a reduced ammonium culture media and an ammonium containing concentrate;
receiving:
the reduced ammonium culture media as a second diluate; and
an electrolyte solution, from an electrolyte solution reservoir, as a second concentrate;
to a second electrodialysis module configured for removing lactate from the reduced ammonium culture media and outputting a reduced ammonium and lactate culture media and a lactate containing solution;
receiving the ammonium containing concentrate to an ion exchange module configured for removing the ammonium and for outputting a reduced ammonium electrolyte solution, and returning the reduced ammonium electrolyte solution to the electrolyte solution reservoir for re-using as concentrate for the first dialysis module and the second dialysis module;
adding an electrolyte in an amount up to 10% v/v to the reduced ammonium and lactate culture media and outputting an electrolyte enriched ammonium reduced and lactate reduced culture media to be returned to the culture media reservoir thereby purifying the spent culture media.

According to some embodiments, the method for recycling spent culture media by removing ammonium and lactate may include:
receiving:
spent culture media as a first diluate, from a culture media reservoir, and
an electrolyte solution, from an electrolyte solution reservoir, as a first concentrate,
to a first electrodialysis module configured for removing ammonium from the received spent culture media and outputting a reduced ammonium culture media and an ammonium containing concentrate;
receiving the reduced ammonium culture media to an ion exchange module configured for removing lactate and for outputting a reduced ammonium and lactate culture media;
receiving the ammonium containing concentrate to an ion exchange module configured for removing the ammonium and for outputting a reduced ammonium solution;
receiving:
the reduced ammonium solution as diluate; and
the reduced ammonium and lactate culture media as a second concentrate to a second electrodialysis module configured for returning the reduced ammonium solution to the reduced ammonium and lactate culture media and for outputting an electrolyte enriched ammonium reduced and lactate reduced culture media to be returned to the culture media reservoir thereby purifying the spent culture media, and
returning an electrolyte solution to the electrolyte solution reservoir for re-using as concentrate for the first dialysis module and the second dialysis module.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the Figures:

FIG. 1: A conceptual scheme of the innovative cultured meat media recycling technology in accordance with some embodiments;

FIG. 2: A box diagram of the overall process for the selective separation of lactate and ammonium from culture media in accordance with some embodiments;

FIG. 3: Illustration of the stack arrangement, process streams and transport of most significant ions in the first electrodialysis module in accordance with some embodiments;

FIG. 4: Illustration of the stack arrangement, process streams and transport of most significant ions in the second electrodialysis module in accordance with some embodiments;

FIG. 5: Illustration of the stack arrangement, process streams and transport of most significant ions in the third electrodialysis module in accordance with some embodiments;

FIG. 6: An exemplary graph showing lactate concentration in the diluate and concentrate and electrical conductivity as a function of time in accordance with some embodiments;

FIG. 7: An exemplary graph showing ammonia concentration in the concentrate and electrode rinse solutions during the electrodialysis experiments as a function of electric current reduction in accordance with some embodiments;

FIG. 8: An exemplary graph showing change in voltage, pH and electric current as a function of time in the diluate of the second electrodialysis module in accordance with some embodiments; and

FIG. 9: An exemplary graph showing ammonium removal from the concentrate output by the first electrodialysis module by ion exchange in accordance with some embodiments.

FIG. 10: An exemplary graph showing ammonium removal from the concentrate by the second electrodialysis module by ion exchange in accordance with some embodiments.

FIG. 11A-B: Exemplary graphs showing the variation of concentration of ammonium (ppm) in the concentrate chamber with time from Stage A and Stage B ED experiments.

FIG. 12A-B: Exemplary graphs showing the variation of concentration of lactate (ppm) in the concentrate chamber with time from Stage A and Stage B ED experiments.

FIG. 13A-B: Exemplary graphs showing the variation of concentration of lactate (ppm) in the diluate and the concentrate chambers with time from Stage A ED experiment.

FIG. 14A-B: Exemplary graphs showing the variation of concentration of lactate (ppm) in the diluate and the concentrate chambers with time from Stage B ED experiment.

FIG. 15A-G: Exemplary graphs showing the variation of concentration of various ions (Cl⁻, Na⁺, SO₄²⁻, K⁺, P, Mg²⁺ and Ca²⁺) during the first and second ED steps.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the following description, various aspects of the invention will be described. For the purpose of explanation, specific details are set forth in order to provide a thorough understanding of the invention. However, it will also be apparent to one skilled in the art that the invention may be practiced without specific details being presented herein. Furthermore, well-known features may be omitted or simplified in order not to obscure the invention.

According to some embodiments, provided herein are systems and methods for recycling culture media by removing undesired material therefrom, which are safe, efficient, cost effective, and able to specifically remove selected undesired materials from culture media, under various conditions and settings. According to some embodiments, the systems and methods may be for the selective removal of growth inhibitors, such as lactate and ammonium, from spent growth media.

According to some embodiments, the systems and methods for recycling culture media may include one or more electrodialysis modules and one or more selective ion-exchange modules. According to some embodiments, two principles of electrodialysis may enable the selective separation (i) the (electric) driving force used may allow separating ions from neutral solutes (ii) the transport rate of ions through the ion-exchange membranes used may depend on ion size, valency and/or affinity to the membrane. According to some embodiments, a design based on these principles may enable the separation of the undesirable materials (e.g., growth inhibitors) while maintaining most of the other vital materials (e.g., growth media components).

According to some embodiments, electrodialysis modules described here may contain a 2-cell repeating unit (e.g., standard electrodialysis cell arrangement), comprising of an Anion Exchange Membrane (AEM) and a Cation Exchange Membrane (AEM). Optionally, each electrodialysis module may contain two diluate (ion depleting) streams and a concentrate (ion enriched) stream, both entering and exiting from the electrodialysis stack as independent streams that may not be in direct contact, which may help reduce the chance contamination.

According to some embodiments, the recycling system for removing at least one undesired material from spent culture media may include one or more culture media reservoir, one or more electrolyte solution reservoir, one or more electrodialysis modules, and one or more ion exchange modules. Optionally, the order of the electrodialysis module/s and ion exchange module/s may be variable.

According to some embodiments, the recycling system may be configured to remove an electrolyte and one or more undesired materials from the spent culture media utilizing the one or more electrodialysis modules and the one or more ion exchange module, to recover a removed electrolyte, and to return the electrolyte to the recycled culture media.

The term “undesired material” as used herein in accordance with some embodiments relates to a compound which may negatively affect the growth and/or well-being of the cultured tissue and/or cells. Non-limiting examples of undesired material are: a growth inhibitor, a waste product, an impurity, a contaminant, a metabolite, an excess of one or more culture media components, and similar and/or a combination thereof.

According to some embodiments, the undesired material may be selected from a group including ammonium, lactate, lactose, hydrogen, alcohol (e.g., ethanol), alkaloid, carbon dioxide, uric acid, urea, creatine, creatinine, an amino acid, or any combination thereof. Each possibility is a separate embodiment.

The term “vital material” as used herein in accordance with some embodiments relates to a compound which may positively affect the growth and/or well-being of the cultured tissue and/or cells. Non-limiting examples of vital material are: a growth factor, an amino acid, a vitamin, a protein, an enzyme, a co-enzyme, a hormone, a sugar, a carbohydrate, a micronutrient, macronutrient, a mineral, an osmolarity agent, a pH maintenance agent, and combinations thereof.

The term “culture media” as used herein in accordance with some embodiments relates to a solution used to cultivate cells and/or tissue, and includes all the vital material for cell cultivation. For example, the cultivated cells and/or tissues may include tissue including animal tissue, plant tissue, fungal, algal tissue, animal cells, plant cells, bacteria, yeast, fungi, microalgae, algae, or any combination thereof. Each possibility is a separate embodiment.

The term “fresh culture media” as used herein in accordance with some embodiments relates to a solution which includes all the vital material for cell and/or tissue cultivation and little to no undesired material.

The term “spent culture media” as used herein in accordance with some embodiments relates to a solution which includes all or some of the vital material for cell and/or tissue cultivation and sufficient undesired material to negatively affect cell and/or tissue cultivation.

The term “recycled culture media” as used herein in accordance with some embodiments relates to a solution which includes all or some of the vital material for cell and/or tissue cultivation and from which all or some of the undesired material has been removed.

According to some embodiments, the recycled culture media may be considered as “fresh culture media”. According to some embodiments, the recycling system may be configured to operate as a continuous process, a semi-batch process, or a batch process. Optionally, the culture medial recycling may be repeated n times, wherein n>1. The term “reduced” as used herein in accordance with some embodiments is defined as decreased. Optionally, a reduced amount may relate to an amount of a compound in a solution which may be decreased. Optionally, the term “reduced” may relate to a reduction of at least about 5%, at least about 10%, at least about 15%, at least about 20%, at least about 25%, at least about 30%, at least about 35%, at least about 40%, at least about 45%, at least about 55%, at least about 60%, at least about 65%, at least about 70%, at least about 75%, at least about 80%, at least about 85%, at least about 90%, or at least about 95%. Each is a separate embodiment.

The term “electrodialysis” as used herein in accordance with some embodiments relates to an electrochemical separation process that uses an electric current to move ions through one or more selective permeable or semi-permeable membranes. Optionally, the electrodialysis may be reverse electrodialysis, for example, the polarity of the electrodes may be periodically reversed. Optionally, one or more membranes may be selective to cations (CEM) and/or to anions (AEM). Optionally, one or more membranes may be selective to valency, size, polarity, etc.

The term “ion exchange” as used herein in accordance with some embodiments relates to a reversible interchange of one type of ion present in an insoluble solid, semi-solid, liquid or membrane, with another ion of like charge present in a solution surrounding the solid, semi-solid, liquid or membrane. Optionally, an ion exchanger may be selected from a membrane, a column, a bed, or suspended beads. Optionally, an ion exchange module may be operated in columns, suspension and separation, or mixed matrix filter.

According to some embodiments, the recycled culture media may be used for cultivated meat production, cultivated plant production, artificial protein production, enzyme production, metabolites (e.g., primary metabolites, secondary metabolites, etc.), growth of artificial organs, and/or production of pharmaceutically active components. According to some embodiments, the culture media may be used to cultivate cells selected from animal tissue culture, plant tissue culture, microorganisms, bacteria, yeast or fungi, microalgae, and/or algae. Each possibility is a separate embodiment. Preferably, the culture media may be used to cultivate cells from animal tissue.

According to some embodiments, the culture media may be used to cultivate cells selected from animal tissue. According to some embodiments, the system may be configured to remove one or more undesired materials (such as, waste products and/or growth inhibitors, etc.) from culture media used to cultivate cells selected from animal tissue. Non-limiting examples of undesired materials include ammonium, lactate, lactose, hydrogen, alcohol (e.g., ethanol), alkaloids, carbon dioxide, uric acid, urea, creatine, creatinine, amino acids, or any combination thereof. Each possibility is a separate embodiment.

According to some embodiments, the culture media may be used to cultivate cells selected from plant tissue. According to some embodiments, the system may be configured to remove one or more undesired materials (such as, waste products and/or growth inhibitors, etc.) from culture media used to cultivate cells selected from plant tissue. Non-limiting examples of undesired materials include ammonium, lactate, lactose, hydrogen, alcohol (e.g., ethanol), alkaloids, carbon dioxide, uric acid, urea, creatine, creatinine, amino acids, or any combination thereof. Each possibility is a separate embodiment. For example, plants may have substances that may be known to be growth inhibitors, such as, certain metabolites which may vary between different crops, and even within the same plant type, between various plant tissues. Optionally, some of these substances may even be cell killers. An example of a growth-inhibiting substance are alkaloids such as solanines found in species of the nightshade family within the genus Solanum.

According to some embodiments, the culture media may be used to cultivate a microorganism, e.g., yeast, bacteria, etc. According to some embodiments, the system may be configured to remove one or more undesired materials (such as, waste products and/or growth inhibitors, etc.) from culture media used to cultivate a microorganism. Non-limiting examples of undesired materials include ammonium, lactate, lactose, hydrogen, alcohol (e.g., ethanol), alkaloids, carbon dioxide, uric acid, urea, creatine, creatinine, amino acids, or any combination thereof. Each possibility is a separate embodiment. For example, yeast growth may be inhibited by alcohol, a metabolite produced naturally by the yeast, whoever, when alcohol is present in excess, the yeast may die. Many methods of regulating nutrients (for example, sugars), temperature, and pH may be used such that the yeast may produce metabolites, which may be of interest in various fields, and may produce less alcohol. Optionally, when alcohol is the desired metabolite, yeast strains that are more resistant to the presence of alcohol may be used, and/or additional nutrients and/or more yeast may be added to continue the production of the alcohol. According to some embodiments, a system of culture media circulation may be used which removes alcohol and recycles the culture media for further use may significantly improve (and simplify the constant control required) the production and/or use of yeast in various industrial processes.

According to some embodiments, the system may be configured to return to the recycled culture media a vital material that was removed during the recycling process, and/or to replace a depleted vital material in the culture media. Optionally, such a vital material may be a growth factor, an amino acid, a vitamin, a protein, an enzyme, a co-enzyme, a hormone, a sugar, a carbohydrate, a micronutrient, macronutrient, a mineral, an osmolarity agent, a pH maintenance agent, and combinations thereof.

According to some embodiments, the recycling system for removing a growth inhibitor from spent culture media may include:
a culture media reservoir;
an electrolyte solution reservoir;
a first electrodialysis module configured to receive, spent culture media from the culture media reservoir as a first diluate; and an electrolyte solution from the electrolyte solution reservoir as a first concentrate, wherein the first electrodialysis module may be configured to remove ammonium from the received spent culture media and to output a reduced ammonium culture media and an ammonium containing concentrate;
a second electrodialysis module configured to receive the reduced ammonium culture media as a second diluate; and an electrolyte solution, from the electrolyte solution reservoir as a second concentrate, wherein the second electrodialysis may be configured to remove lactate from the reduced ammonium culture media and to output a reduced ammonium and lactate culture media and a lactate containing solution;
an ion exchange module configured to receive the ammonium containing concentrate, to remove the ammonium and to output a reduced ammonium solution, whereas the ammonium was exchanged for another cation (e.g., Na+, K+, H+, etc.); and
a third electrodialysis module configured to receive the reduced ammonium solution as diluate; and the reduced ammonium and lactate culture media as a third concentrate, wherein the third electrodialysis module may be configured to return the reduced ammonium solution to the reduced ammonium and lactate culture media; and
to output an electrolyte enriched ammonium and lactate reduced culture media to be returned to the culture media reservoir thereby purifying the spent culture media, and an electrolyte solution to be returned to the electrolyte solution reservoir for re-use as concentrate for the first and second dialysis modules.

According to some embodiments, the recycling system for removing a growth inhibitor from spent culture media may include:
a culture media reservoir;
an electrolyte solution reservoir;
a first electrodialysis module configured to receive spent culture media from the culture media reservoir as a first diluate; and an electrolyte solution from the electrolyte solution reservoir as a first concentrate, wherein the first electrodialysis module may be configured to remove ammonium from the received spent culture media and to output a reduced ammonium culture media and an ammonium containing concentrate;
a second electrodialysis module configured to receive the reduced ammonium culture media as a second diluate; and an electrolyte solution from the electrolyte solution reservoir as a second concentrate, wherein the second electrodialysis may be configured to remove lactate from the reduced ammonium culture media and to output a reduced ammonium and lactate culture media and a lactate containing solution;
an ion exchange module configured to receive the ammonium containing concentrate, to remove the ammonium and to output a reduced ammonium solution which may be returned to the electrolyte solution reservoir for re-use as concentrate for the first dialysis module and second dialysis modules;
an electrolyte may be added to the reduced ammonium and lactate culture media to make up for the electrolyte lost in the first electrodialysis module and to output an electrolyte enriched ammonium and lactate reduced culture media to be returned to the culture media reservoir thereby purifying the spent culture media.

According to some embodiments, the recycling system for removing a growth inhibitor from spent culture media may include:
a culture media reservoir;
an electrolyte solution reservoir;
a first electrodialysis module configured to receive, spent culture media from the culture media reservoir as a first diluate; and an electrolyte solution from the electrolyte solution reservoir as a first concentrate, wherein the first electrodialysis module may be configured to remove ammonium from the received spent culture media and to output a reduced ammonium culture media and an ammonium concentrate;
a first ion exchange module configured to receive the reduced ammonium culture media, to remove lactate and to output a reduced ammonium and lactate culture media;
a second ion exchange module configured to receive the ammonium containing concentrate, to remove the ammonium and to output a reduced ammonium solution; and
a second electrodialysis module configured to receive the reduced ammonium solution as diluate; and the reduced ammonium and lactate culture media as a third concentrate, wherein the second electrodialysis module may be configured to return the reduced ammonium solution to the reduced ammonium and lactate culture media; and
to output an electrolyte enriched ammonium and lactate reduced culture media to be returned to the culture media reservoir thereby purifying the spent culture media, and an electrolyte solution to be returned to the electrolyte solution reservoir for re-use as concentrate for the first dialysis module.

According to some embodiments, the amount of electrolyte added to the reduced ammonium and lactate culture media may be up to about 0.1% v/v, up to about 1% v/v, up to about 5% v/v, up to about 10% v/v, or up to about 15% v/v.

According to some embodiments, the electrolyte may be a salt solution including sodium, calcium, potassium, chloride, phosphate, carbonate, bicarbonate, magnesium, sulfonic acid, sulfate, nitrate, organic salts like acetate, citrate, formate, a derivative thereof, or a combination thereof. Optionally, the electrolyte may be a NaCl solution. According to some embodiments, the electrolyte concentration in the electrolyte may be controlled by adjusting current density, voltage, and hydraulic residence time.

According to some embodiments, the one or more electrodialysis modules may include a membrane selective for an undesired material. According to some embodiments, the one or more electrodialysis modules may include one or more 2-cell repeating units. According to some embodiments, a 2-cell repeating unit may include an Anion Exchange Membrane (AEM) and a Cation Exchange Membrane (CEM). Optionally, spacers may be located between the CEM and AEM. According to some embodiments, the number of 2-cell repeating unit may be in the range between about 1-300 repeating units.

According to some embodiments, the AEM of one or more electrodialysis modules may be configured to be non-selective, partially selective, and/or selective. Optionally, the selectivity may be based on charge, ion type, size, valency, polarity, etc. and/or any combination thereof.

According to some embodiments, the CEM of one or more electrodialysis modules may be configured to be non-selective, partially selective, and/or selective. Optionally, the selectivity may be based on charge, ion type, size, valency, polarity, etc. and/or any combination thereof.

For example, the AEM of the first electrodialysis module may be configured to allow passage of small negatively charged ions, while the CEM of the first electrodialysis module may be configured to be monovalent cation selective and/or may be configured to allow passage of cations smaller than about 200 Da.

For example, the AEM of the second electrodialysis module may be configured to be selective to lactate, while the CEM of the second electrodialysis module may be configured to allow passage of small positively charged ions.

According to some embodiments, the AEM membrane surface area of one or more electrodialysis modules may be in the range between about 2 cm² up to 2 m² for one repeating unit. According to some embodiments, the CEM membrane surface area of one or more electrodialysis modules may be in the range between about 2 cm² up to 2 m² for one repeating unit.

According to some embodiments, the ion concentration in each of the streams of the one or more electrodialysis modules may be controlled by adjusting current density, voltage, and hydraulic residence time. According to some embodiments, hydraulic residence time of the one or more electrodialysis modules may be in the range between about 30 seconds to 6 hrs, between about 3 mins to 4 hrs, or between about 30 mins to 2 hrs. According to some embodiments, the current density of the one or more electrodialysis modules may be in the range between about 0.1-2000 A/m², between about 1-1000 A/m², or between about 10-500 A/m².

According to some embodiments, about 75-100%, about 80-99% or about 85-95% of the ammonium ions may migrate from the diluate to the concentrate of one or more electrodialysis modules to output a reduced ammonium culture media. Each possibility is a separate embodiment.

According to some embodiments, up to about 90%, up to about 95% or up to about 99% of the lactate may be removed from a reduced ammonium culture media in one or more electrodialysis modules to output a reduced ammonium and lactate culture media. Each possibility is a separate embodiment. Optionally, the output lactate containing solution may be further concentrated for disposal or reuse. Optionally, other materials such as sulphate and/or phosphate may be further separated from the output lactate containing solution and may be recycled back to the growth media. Optionally, this may be facilitated by various separation methods and/or their combinations, including membrane filtration, electrodialysis, adsorption, ion-exchange. Optionally, the pH may be adjusted at this stage.

According to some embodiments, one or more ion exchange modules may include an ion exchanger selected from the group including: a membrane, a column, a bed, or suspended beads. According to some embodiments, an ion exchange module may be operated in columns, suspension and separation, or mixed matrix filter.

According to some embodiments, the ion exchanger may be selective for an undesired material. Optionally, the ion exchanger may have high selectivity to ammonium or lactate. For example, the ion exchange module may include a cation exchange resin with a high affinity towards ammonium, and/or an anion exchange resin with a high affinity towards lactate. According to some embodiments, an ion exchange module may include a zeolite, copper-based resin polysulphone based, polymer-based, or zinc hexacyanoferrate as the ion exchanger. Each possibility is a separate embodiment.

According to some embodiments, up to about 90%, up to about 95% or up to about 99% of the ammonium may be removed from the ammonium containing concentrate in the ion exchange module to output a reduced ammonium solution. Each possibility is a separate embodiment.

According to some embodiments, the recycling system may be configured to repeat each of the modules of growth inhibitor removal and salt recovery n times, wherein n>1.

Reference is now made to the figures.

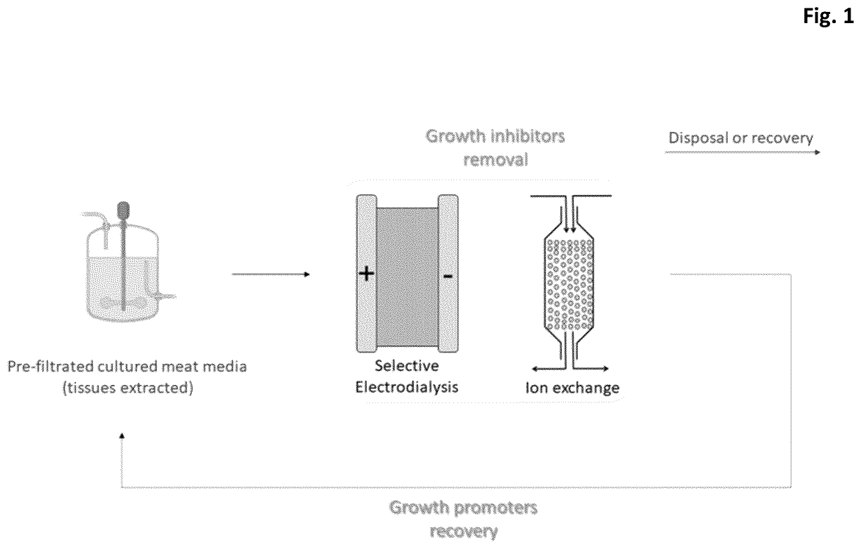

FIG. 1 is a conceptual scheme of the innovative cultured meat media recycling technology in accordance with some embodiments. For example, the proposed process includes growth media removal and growth promoter recovery using a combination of electrodialysis and ion exchange.

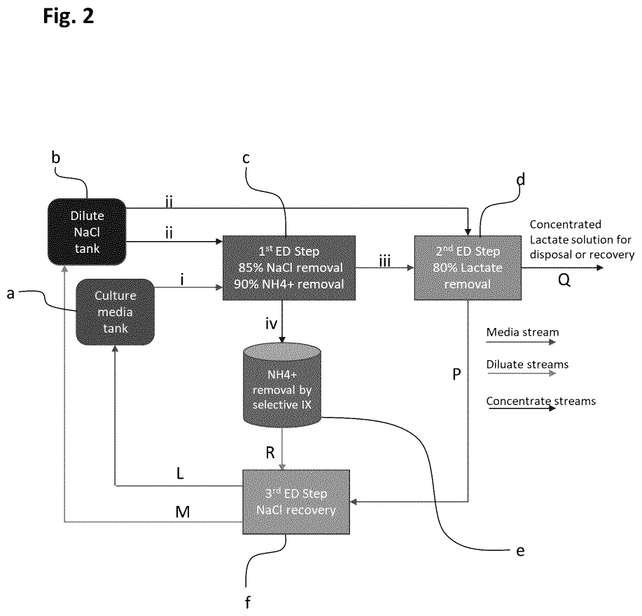

The process exemplified in FIG. 2 is a box diagram of a process for the selective separation of lactate and ammonium from culture media in accordance with some of the embodiments. The process exemplified in FIG. 1 includes three electrodialysis modules and one selective ion-exchange module. For example, the process includes three electrodialysis modules, each contains a diluate stream (ion depleting) and a concentrate stream (ion enriched). The culture media is the diluate in the first and the second electrodialysis modules and the concentrate in the third module. The concentrate of the first electrodialysis module undergoes a selective ion-exchange process to remove ammonium and enter the third electrodialysis module as the diluate. Optionally, it may be operated in a batch, semi-batch, or continuous mode.

For example, the recycling system for removing a growth inhibitor from spent culture media, the system may include:
(a) a culture media reservoir;
(b) an electrolyte solution reservoir;
(c) a first electrodialysis module configured to receive:
   (i) spent culture media from the culture media reservoir as a first diluate; and
   (ii) an electrolyte solution from the electrolyte solution reservoir as a first concentrate,
wherein the first electrodialysis module is configured to remove ammonium from the received spent culture media (i) and to output a reduced ammonium culture media (iii) and an ammonium containing concentrate (iv);
(d) a second electrodialysis module configured to receive:
   (iii) the reduced ammonium culture media as a second diluate; and
   (ii) an electrolyte solution, from the electrolyte solution reservoir (b) as a second concentrate,
wherein the second electrodialysis (d) is configured to remove lactate from the reduced ammonium culture media (iii) and to output a reduced ammonium and lactate culture media (P) and a lactate containing solution (Q);
(e) an ion exchange module configured to receive the ammonium containing concentrate (iv), to remove the ammonium and to output a reduced ammonium solution (R); and
(f) a third electrodialysis module configured to receive:
   (v) the reduced ammonium solution (R) as diluate; and
   (vi) the reduced ammonium and lactate culture media (P) as a third concentrate,
wherein the third electrodialysis module (f) is configured to return the reduced ammonium solution (R) to the reduced ammonium and lactate culture media (P); and
to output:
   an electrolyte enriched, ammonium reduced and lactate reduced culture media (L) to be returned to the culture media reservoir (a) thereby purifying the spent culture media, and
   an electrolyte solution (M) to be returned to the electrolyte solution reservoir (b) for re-use as concentrate for the first dialysis module (c) and second dialysis module (d).

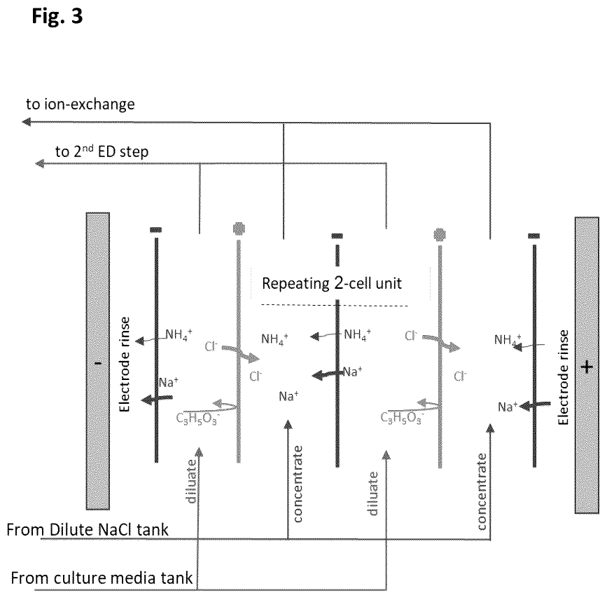

FIG. 3 exemplifies a first electrodialysis module, illustrating the stack arrangement, process streams and transport of most significant ions in accordance with some embodiments. For example, the first electrodialysis module receives spent culture media as diluate and 0.01M NaCl solution as concentrate from the electrolyte reservoir. In this module, the smallest ions, mostly Na⁺, Cl⁻ and NH₄⁺ (optionally, other small ions such as bicarbonate, etc.) were transferred from the spent media to the concentrate. Most vital materials (e.g., hormones, proteins, vitamins, etc.) remain in the media/diluate due to their lack of charge or larger size. Lactate mostly stays in the diluate after the first module due to its larger size. With the right combination of operational parameters (AEM type, membrane surface area, hydraulic residence times, and current density), 75-90% of the small ions, but only 5-25% of the lactate migrate from the diluate to the concentrate. The AEM may be a relatively dense membrane that can block lactate while passing smaller ions like chloride and/or bicarbonate. The salt and ammonium enriched concentrate exiting the first electrodialysis module continues to the ion-exchange module. The ideal CEM properties will depend on the selectivity of the ion exchange resin toward ammonia, as detailed below.

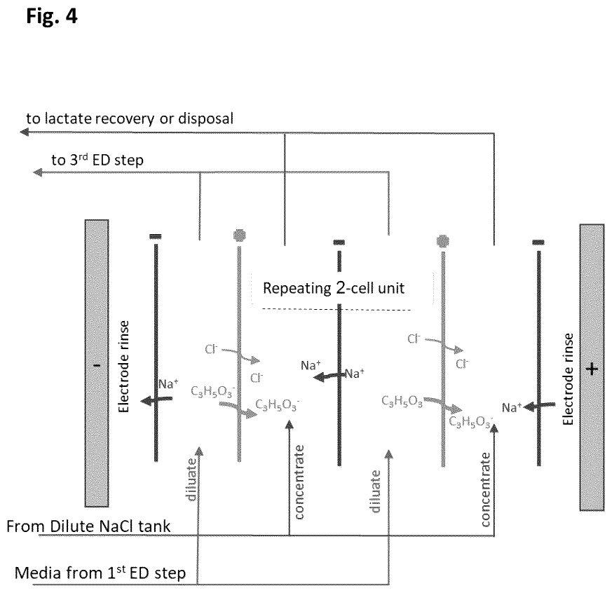

FIG. 4 exemplifies a second electrodialysis module, illustrating the stack arrangement, process streams and transport of most significant ions in accordance with some embodiments. For example, the diluate entering the second electrodialysis module is the spent media exiting the first electrodialysis module (also diluate), while the concentrate is a fresh 0.01M NaCl solution from the electrolyte reservoir. In this module, the media is already depleted of the smaller anions; thus, lactate migrates to the concentrate under the electric field. Up to 95% of the lactate can be removed, while uncharged and large vital material will remain in the diluate (media) stream. By adjusting the concentrate volume (batch mode) or flow rate (continuous mode), the lactate can be concentrated for more convenient disposal or reuse (e.g., in other bio-processes).

In the second module, there is a risk of losing some small valuable charged molecules such as ionized amino acids, hydrogen phosphate ions, SO₄²⁻Ca²⁺, Mg²⁺, and other trace cations like copper and iron. Therefore, the AEM for this module may preferably block all anions from migrating to the concentrate, except for lactate. The CEM for the second electrodialysis module is relatively size and monovalent selective, blocking large (e.g., cationic amino acids) and multivalent (e.g., metals) cations. Nevertheless, any AEM and CEM can be used, with implications on case-specific cost-efficiency. Process conditions can also be manipulated to increase selectivity and may be optimized for each case.

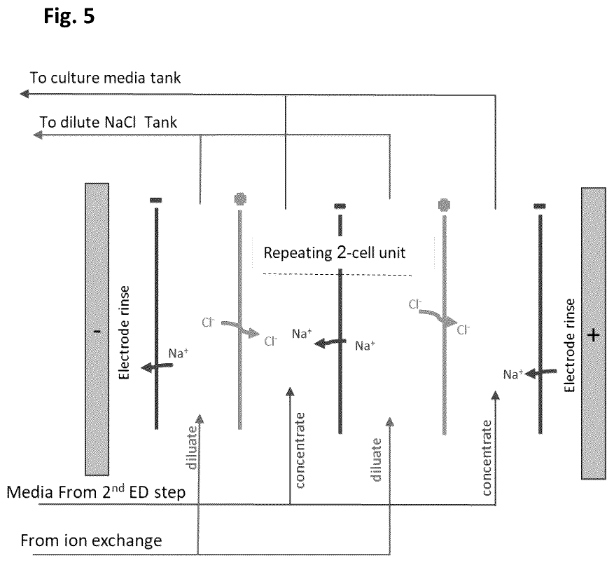

FIG. 5 exemplifies a third electrodialysis module, illustrating the stack arrangement, process streams and transport of most significant ions in accordance with some embodiments. For example, the salt removed from the media in the first electrodialysis module may be returned to it. Electrodialysis may be used to ensure that no excess fluid is added to the recycled culture media. However, this module may be replaced by addition of fresh electrolyte solution from the electrolyte reservoir and/or addition of a solid salt or salt solution from an external source. The salt may be returned to the recycled culture media to maintain osmolarity.

In the third electrodialysis module, the treated media (second electrodialysis module diluate) is used as the concentrate stream in the third electrodialysis module, while the first electrodialysis module concentrate becomes the diluate entering the third electrodialysis module (after ammonium removal by ion-exchange, as detailed below). Ions then migrate from diluate to concentrate under the applied electric field. Both AEM and CEM may be non-selective in this module to allow fast and effective migration of salt ions back to the medium

In the ion-exchange module, a cation exchange resin with a high affinity towards NH4⁺ is applied to remove the NH4⁺ from the concentrate of the first electrodialysis module. Any selective resin may be used for this purpose (e.g., zeolite or copper-based), and the process may be operated in columns, suspension and separation, or mixed matrix filter. However, the efficiency may decline at high salt concentrations if the resin is not selective enough for NH4⁺ over Na⁺. Moreover, if the affinity towards divalent metal cations is high, blocking their passage in the first electrodialysis module may be beneficial by using monovalent selective cation exchange membranes. A non-selective CEM in the first electrodialysis module may be preferred if the resin is very selective. These factors are case-specific and may be optimized.

In the description and claims of the application, the words “include” and “have”, and forms thereof, are not limited to members in a list with which the words may be associated.

As used herein, the term “about” may be used to specify a value of a quantity or parameter (e.g., the length of an element) to within a continuous range of values in the neighborhood of (and including) a given (stated) value. According to some embodiments, “about” may specify the value of a parameter to be between 80% and 120% of the given value.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although modules of methods according to some embodiments may be described in a specific sequence, methods of the disclosure may include some or all of the described modules carried out in a different order. A method of the disclosure may include a few of the modules described or all of the modules described. No particular module in a disclosed method is to be considered an essential module of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

The following examples are presented to provide a more complete understanding of the invention. The specific techniques, conditions, materials, proportions and reported data set forth to illustrate the principles of the invention are exemplary and should not be construed as limiting the scope of the invention.

### EXAMPLES

### Example 1

### Electrodialysis Experiments

Electrodialysis experiments for the first and second electrodialysis modules were carried out using a Belectrodialysis-1-4 experimental system (PCCell Gmbh) with an active membrane area of 64 cm². The treated stream was a commercially available growth medium (DMEM/F12 (1:1) w/HEPES w/o Phenol red by Rhenium, catalog number-11039021), spiked with ammonium (5 mM) and lactate (2 g/l). The pH was adjusted to 7.4 using NaOH/HCl. The electrical current was kept constant at 0.3 A, and the temperature was kept constant at about 38 C. The anion exchange membrane (AEM) was PC-100-D and cation exchange membrane (CEM) was PC—SK, both from PCCell Gmbh. The stack consisted of five cell pairs. The concentrate contained 1 L of 0.01M NaCl, and the electrode rinse contained 1 L of 0.25M sodium sulfate.

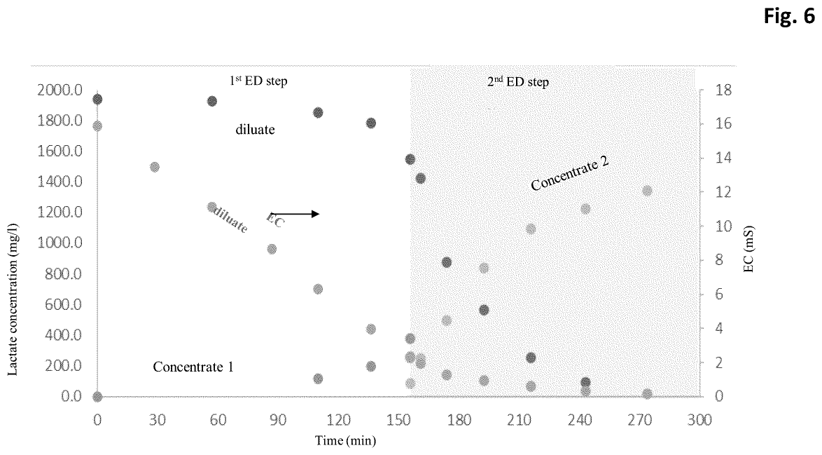

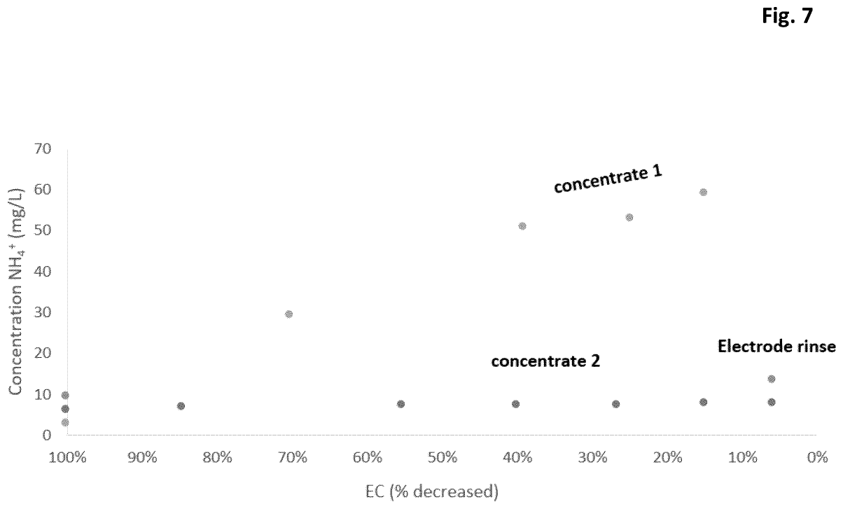

FIG. 6 shows the lactate concentration in the diluate and concentrate (right axis) and electrical conductivity (left axis) as a function of time.

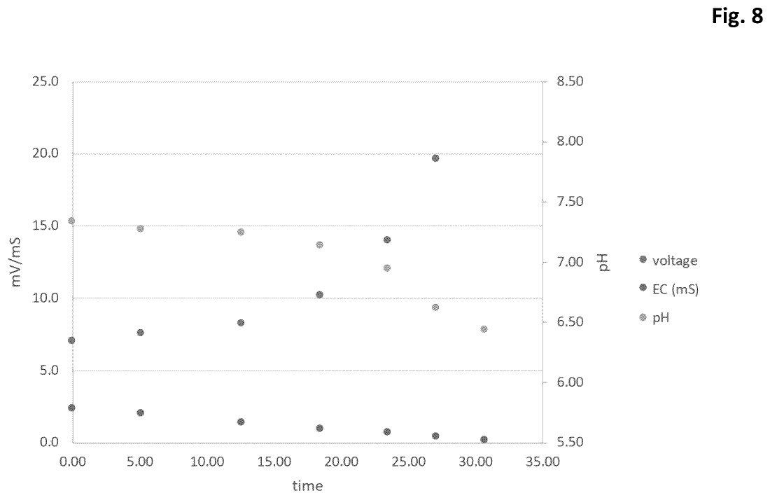

The experiment was performed in 2 stages. In the first module, 85% decline in initial EC of diluate. After stopping the process, the concentrate solution was collected and replaced with a fresh one, while the diluate and electrolyte solutions remained the same. In the second module, after concentrate solution replacement, the process started again as the second electrodialysis module, until 94% decline in initial electrical conductivity (EC) was reached. Lactate concentration was analyzed using HPLC and ammonium concentration was analyzed using the Nessler reagent method. The results demonstrate that both electrodialysis modules worked as expected. After 150 minutes, about 85% of the salt (measured as EC) was removed, while the lactate mainly remained in the diluate. In the second module, the lactate rapidly migrated into the concentrate stream, reaching about 95% removal in the end. In contrast, the ammonium was removed from the medium mainly in the first electrodialysis module, as revealed by a mass balance, using the ammonium concentrations measured in the concentrate and electrode rinse streams. FIG. 7 shows the ammonium concentration in the concentrate and electrode rinse solutions during the electrodialysis experiments as a function of electrical conductivity reduction.

While the pH and voltage were relatively stable in the first electrodialysis module, at the end of the second electrodialysis module, the voltage rapidly increased, and the pH decreased. FIG. 8 shows the change in voltage, pH and electrical conductivity as a function of time in the diluate of the second electrodialysis module.

The increase in voltage was due to a critical reduction in diluate electrical conductivity. The decrease in pH could have been due to water splitting upon reaching the limiting current density or a result of weak bases like phosphate, HEPES, or bicarbonate migrating to the concentrate. This undesired pH change could be addressed for a given case by adjusting the current and selection of the membranes.

### Ion Exchange for Ammonium Removal

Ammonium removal experiments were carried out in batch mode using a zinc hexacyanoferrate (Zn—HCF) inorganic ion exchanger. Zn—HCF was prepared by following the procedure reported in the literature (Nativ et al., 2021; Takahashi et al., 2016). During batch adsorption experiments, 25 ml of each concentrate solution (collected after first electrodialysis module (stage A) and second electrodialysis module (Stage B)) was added into 50 ml falcon tubes. Then, different doses of Zn—HCF were added. These Falcon tubes were stirred in an incubator shaker at 200 rpm stirring speed for 36 h while the temperature was maintained at 37.5° C. After 36 h, Zn—HCF particles were separated from the solution, and initial (C0) and equilibrium concentrations (Ce) of ammonium ions were measured using the spectrophotometric method. Ammonium removal (%) and ammonium removal capacity of Zn—HCF was estimated using the following equations.

$$Ammonium removal ( \% ) = \frac{C 0 - Ce}{C 0} \times 100 \qquad ( 1 )$$

$$Ammonium removal capacity ( mg / g ) = \frac{C 0 - Ce}{M} \times V \qquad ( 2 )$$

Where C0, Ce, M, and V are initial ammonium concentration (mg/L), equilibrium ammonium concentration (mg/L) after the ion exchange process, and the mass of adsorbent (g) and Volume (Liter), respectively.

TABLE 1
Characteristics of ED experiments concentrate
solutions after Stage A and Stage B

| | Concentrate A | Concentrate B |
|---|---|---|
| Characteristics | (after Stage A) | (after Stage B) |
| Ammonium | 69.17 | 11.0 |
| concentration as N | | |
| (mg/L) | | |
| pH | 7.1 | 8.6 |
| Conductivity (mS) | 26.9 | 6.19 |

Conductivity, pH and concentration of ammonium ions in solutions (after stage A and stage B) collected after ED experiments have been shown in Table 1. The electrodialysis process separated most of the ammonium ions in the first electrodialysis module into the concentrate stream (A). Concentrate A comprises high conductivity (about 27 mS) and ammonium ions (79.17 mg/L). Zn—HCF adsorbent was used to remove ammonium.

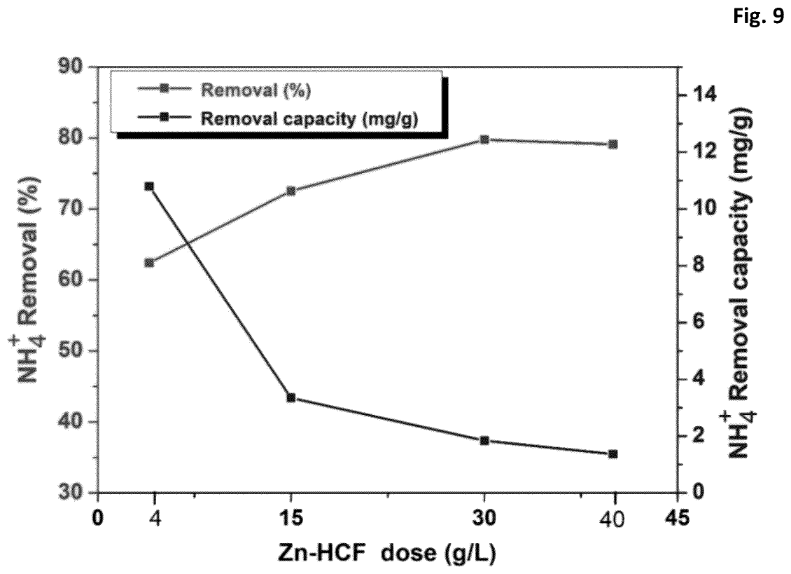

FIG. 9 shows ammonium removal from ED experiment concentrate (after stage A), the experimental conditions were Ammonium concentration: 69.17 mg/L, Zn—HCF dose: 4 to 40 g/L, Solution conductivity: 26.9 mS, Solution pH: 7.1, Temperature: 37.5° C., stirring speed: 200 rpm, Stirring time: 36 h.

FIG. 9 shows that about 80% ammonium removal was achieved at 30 g/L resin concentration. Afterward, no significant increase in ammonium removal was observed at higher concentrations. This can be attributed to the fact that a higher dose of ion exchanger at fixed ammonium concentration provides a large no of ion exchange site, but because of the diffusional control of the ion exchange process, after an optimum dose, further increase in Zn—HCF quantities does not increases the ammonium removal performance. This observation agreed with earlier studies (Hekmatzadeh et al., 2013; Sica et al., 2014).

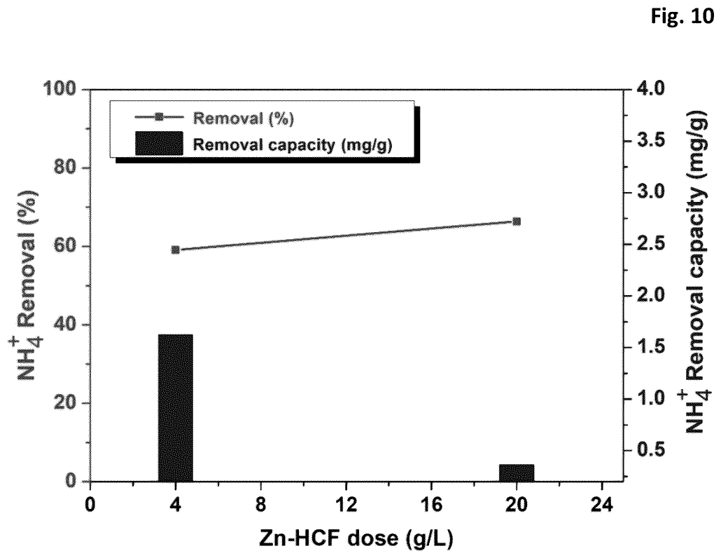

FIG. 10 shows ammonium removal from ED experiment concentrate (after stage B), the experimental conditions were Ammonium concentration: 11 mg/L, Zn—HCF dose: 4 & 20 g/L, Solution conductivity: 6.19 mS, Solution pH: 8.6, Temperature: 37.5° C., stirring speed: 200 rpm, Stirring time: 36 h.

A similar trend was also observed for Stage B concentrate solution which contains lower conductivity (6.19 mS) and ammonium concentration (˜ 11 mg/L) than concentrate Stage A solution. At 4 g/L, ˜ 60% removal was achieved but increase in Zn—HCF dose by several fold (20 g/L) does not increase the removal performance significantly (ammonium removal at 20 g/L dose ˜66%) (FIG. 10).

Ammonium removal results show that Zn—HCF has high selectivity towards ammonium ions. Zn—HCF based adsorptive column/membrane-based technologies could be integrated with electrodialysis process to remove excess toxic ammonium ions from real culture media.

Overall, the ammonium removal results show that Zn—HCF has high selectivity towards ammonium ions. The results indicate that Zn—HCF adsorbent has practical utility, and suggest that Zn—HCF-based ion exchange column/membrane-based technologies can be integrated with electrodialysis process to remove excess toxic ammonium ions from real culture media.

### Example 2

### Electrodialysis Experiments

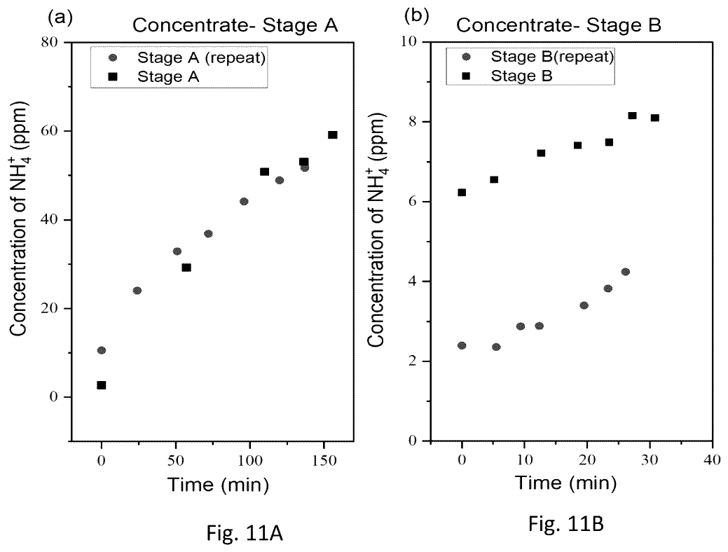

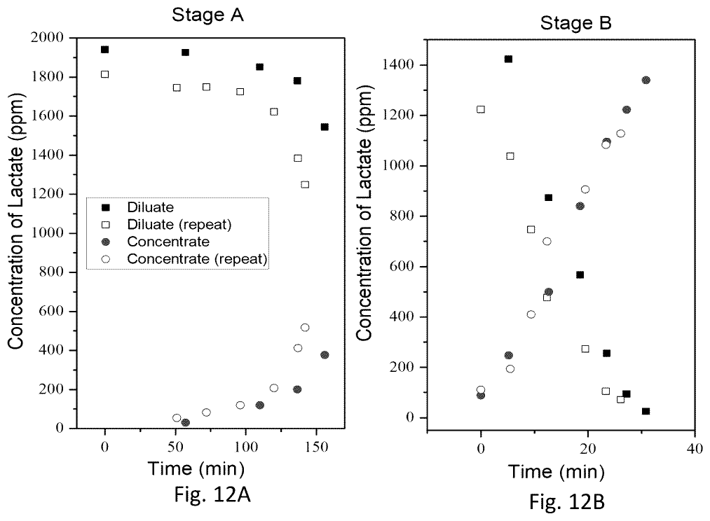

Using two-stage electrodialysis (ED), ammonium and lactate ions were removed from fresh growth medium spiked with ammonium and lactate. For this purpose, 5 mM ammonium chloride and 2000 mg·L⁻¹ lactic acid was added into 1 L of fresh growth medium and adjusted the pH to ˜7.4. In Stage A of ED experiments, growth medium with ammonium and lactate was used as diluate and 0.15M NaCl as concentrate solution. The experiment was carried out at a constant current of 0.3 A, temperature 37.5° C. and flow rate ˜30 LPH until 85% reduction of electrical conductivity (EC). In Stage A of ED, it was expected that the inorganic ions (NH₄⁺ and Cl⁻) along with small amounts of organics (lactate and amino acids) would be transported to the concentrate chamber. Samples were collected at regular intervals, and the concentration of ammonium and lactate in the diluate and concentrate chambers were analyzed using Nesseler method and HPLC, respectively (shown in FIG. 11 and FIG. 12). After the Stage A experiment, the concentrate solution was taken out and ammonium removal experiments carried out separately with various adsorbents. In order to further remove the lactate ions from diluate, a second module ED (Stage B) was required. For Stage B ED experiments, feed left after stage A continued further as the diluate and a fresh solution of 0.01M NaCl was taken as the concentrate. Stage B experiment was carried out at a constant current of 0.3 A, temperature 37.5° C. and flow rate ˜30 LPH until 95-98% reduction of EC. Samples of diluate and concentrate from Stage B experiments were collected and analyzed with respect to the concentrations of ammonium and lactate ions. The two stage ED experiments were duplicated to check repeatability.

FIG. 11 is an exemplary graph showing the variation of concentration of ammonium (ppm) in the concentrate chamber with time from the first electrodialysis module (Stage A) and the second electrodialysis module (Stage B) ED experiments. Stage A ED experiments were carried out with diluate: fresh growth medium+5 mM ammonium chloride+2000 mg·L⁻¹ lactic acid (of volume 1 L, PH ˜7.4); concentrate: 0.15M NaCl at a constant current of 0.3 A, temperature 37.5° C. and flow rate ˜30 LPH. Stage B ED experiments were carried out with diluate: Stage A diluate continued; concentrate: 0.01M NaCl at a constant current of 0.3 A, temperature 37.5° C. and flow rate ˜30 LPH.

As seen in FIG. 11, the concentration of ammonium ion in the concentrate chamber from Stage A (FIG. 11A) and Stage B (FIG. 11B) of ED increased with time. By comparing the concentrations in both the stages, it could be seen that most of the ammonium was transported in Stage A and the remaining amount in Stage B. In both the repetitions, similar trends were observed.

FIG. 12 shows the variation of concentration of lactate (ppm) in the concentrate chamber with time from Stage A and Stage B ED experiments. Stage A ED experiments were carried out with diluate: fresh growth medium+5 mM ammonium chloride+2000 mg·L⁻¹ lactic acid (of volume 1 L, PH ˜7.4); concentrate: 0.15M NaCl at a constant current of 0.3 A, temperature 37.5° C. and flow rate ˜30 LPH. Stage B ED experiments were carried out with diluate: Stage A diluate continued; concentrate: 0.01M NaCl at a constant current of 0.3 A, temperature 37.5° C. and flow rate ˜30 LPH.

The concentration of lactate ion in the diluate and concentrate chambers from Stage A and Stage B of ED were analyzed and shown in FIG. 12A and FIG. 12B. Over time, the concentration of lactate ion decreases in the diluate chamber and increases in the concentrate chamber (i.e., lactate was transported from diluate to concentrate chamber). From Stage A ED (FIG. 12A), the amount of lactate transported from the diluate to the concentrate chamber was only ˜25-35%. This could be due to the transport competition between inorganics and organic ions and/or favorable transport of higher amounts of inorganics through the ion exchange membranes during the ED process. It was observed that most of the lactate was removed in the Stage B ED experiment (FIG. 12B). A clear trend in removal of lactate was noticed in both the repeated experiments. Using two stage ED, lactate ion removal from growth medium was successful.

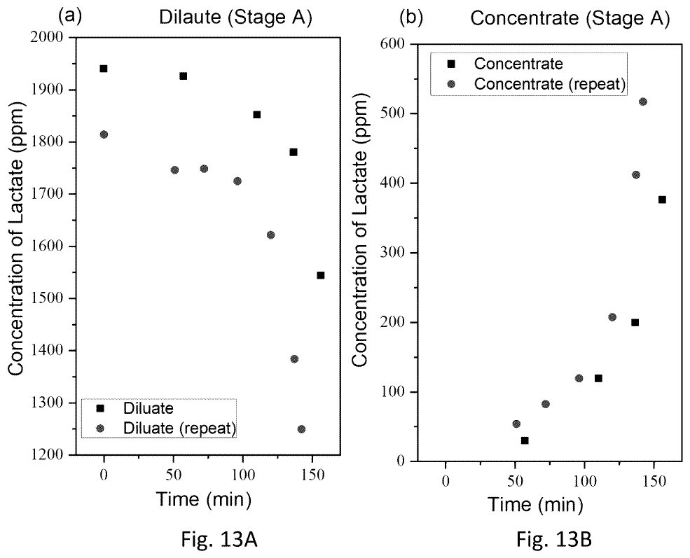

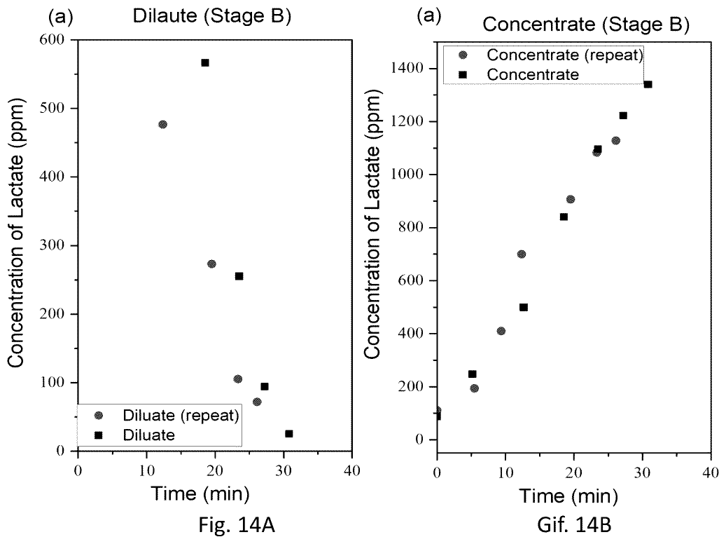

Similarly, FIG. 13 shows variation of concentration of lactate (ppm) in the diluate and the concentrate chambers with time from Stage A ED experiment. The Stage A ED experiments were carried out with diluate: fresh growth medium+5 mM ammonium chloride+2000 mg· L⁻¹ lactic acid (of volume 1 L, PH ˜7.4); concentrate: 0.15M NaCl at a constant current of 0.3 A, temperature 37.5° C. and flow rate ˜30 LPH.

Additionally, FIG. 14 shows variation of concentration of lactate (ppm) in the diluate and the concentrate chambers with time from Stage B ED experiment. The Stage B ED experiments were carried out with diluate: Stage A diluate continued; concentrate: 0.01M NaCl at a constant current of 0.3 A, temperature 37.5° C. and flow rate ˜30 LPH.

### Other Inorganic Ions

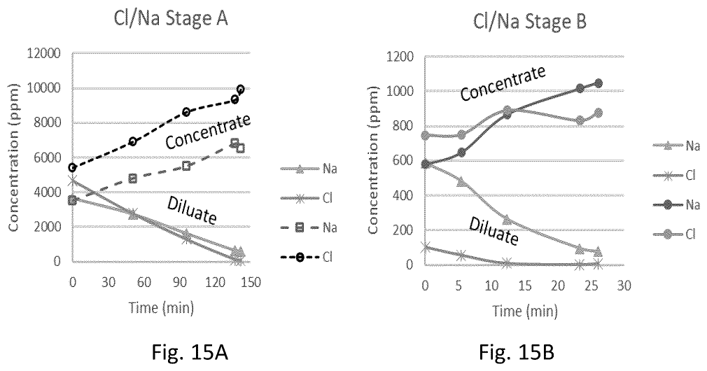

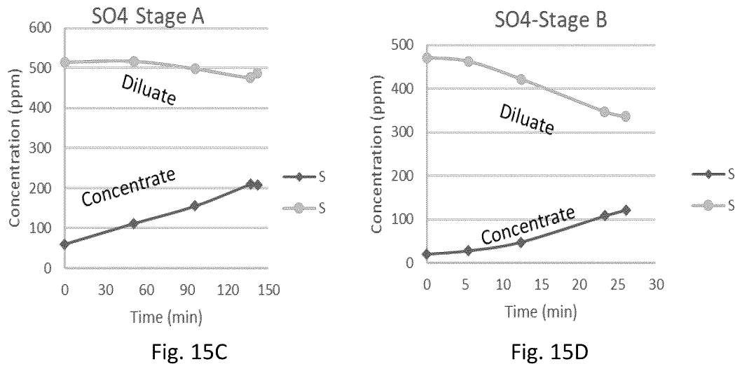

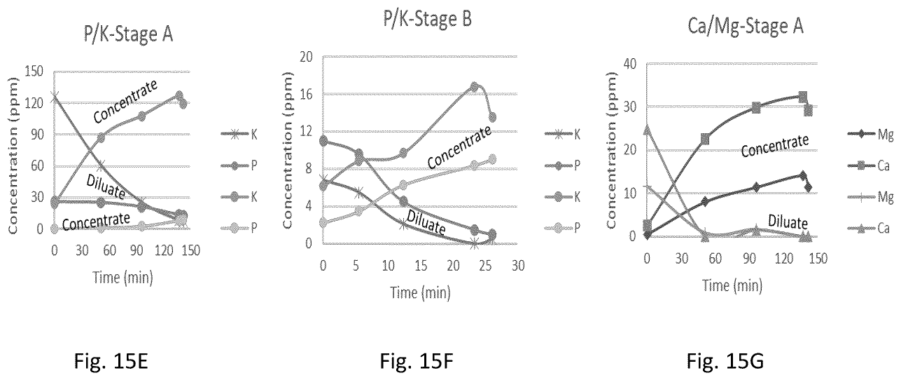

To track other major inorganic ions, samples at different time intervals were collected and measured concentrations using ICP-OES. The results are shown in FIG. 15. Na⁺ and Cl-concentrations declined sharply in the first stage diluate, confirming the EC measurements. At the second stage, Na⁺ and Cl-concentrations almost reached zero concentration in the diluate. Accordingly in both stages, Na⁺ and Cl-pass to the concentrate. Therefore, most of the NaCl was recovered in the process.

This was also true for K⁺, Mg²⁺ and Ca²⁺, which mostly passed to the first stage ED concentrate and was recovered after ammonium was removed. In contrast, the SO₄²⁻ mostly stayed in the first stage concentrate, and passed partially in the second step to the concentrate. According to these results, ˜33% of the SO₄²⁻ ions could end up in the waste stream (second stage concentrate) with the lactate. Moreover, only 50% of the total phosphate (P) passed to the first ED concentrate (and may be recovered after ammonium removal), while in the second stage, most of the phosphate ends up in the waste stream.

FIG. 15 Concentrations of Cl⁻, Na⁻, SO₄²⁻, K⁺, P, Mg²⁺ and Ca²⁺ during the first and second ED steps (Stage A and Stage B, respectively). All concentrations were measured using ICP-OES. FIG. 15A and FIG. 15B show the concentrations of Cl⁻ and Na⁺ in Stage A and Stage B, respectively, FIG. 15C and FIG. 15D show the concentration of SO₄²⁻ in Stage A and Stage B, respectively, FIG. 15E and FIG. 15F show the concentrations of P and K⁺ in Stage A and Stage B, respectively, and FIG. 15G shows the concentration of Mg²⁺ and Ca²⁺ in Stage A.

The results show that Na⁺, Cl⁻, K⁺, Mg²⁺ and Ca²⁺, were easily separated from lactate by the ED and could be recycled by the process suggested herein. For SO₄²⁻ and P, further optimization may be needed, which may include using different anion exchange membranes for the two ED stages, adjusting the temperature and/or the current density. These elements could be recovered from the waste streams by various methods, such as, ion-exchange, membrane filtration, adsorption or ED at a different pH (which is acceptable in the waste stream but not in the media).

### REFERENCES

[1] Hekmatzadeh, A. A., Karimi-Jashni, A., Talebbeydokhti, N., & Kløve, B. (2013). Adsorption kinetics of nitrate ions on ion exchange resin. Desalination, 326, 125-134. https://doi.org/10.1016/j.desal.2013.07.017
[2] Nativ, P., Ben-Asher, R., Fridman-Bishop, N., & Lahav, O. (2021). Synthesis and characterization of zinc-hexacyanoferrate composite beads for controlling the ammonia concentration in low-temperature live seafood transports. Water Research, 203. https://doi.org/10.1016/j.watres.2021.117551
[3] Sica, M., Duta, A., Teodosiu, C., & Draghici, C. (2014). Thermodynamic and kinetic study on ammonium removal from a synthetic water solution using ion exchange resin. Clean Technologies and Environmental Policy, 16 (2), 351-359. https://doi.org/10.1007/s10098-013-0625-3
[4] Takahashi, A., Kitajima, A., Parajuli, D., Hakuta, Y., Tanaka, H., Ohkoshi, S. ichi, & Kawamoto, T. (2016). Radioactive cesium removal from ash-washing solution with high pH and high K⁺-concentration using potassium zinc hexacyanoferrate. Chemical Engineering Research and Design, 109, 513-518. https://doi.org/10.1016/j.cherd.2016.02.027

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. A recycling system for removing a growth inhibitor from spent culture media, the system comprising:
(a) a culture media reservoir,
(b) an electrolyte solution reservoir,
(c) a first electrodialysis module configured to receive:
(i) spent culture media from the culture media reservoir as a first diluate; and
(ii) an electrolyte solution from the electrolyte solution reservoir as a first concentrate,
wherein the first electrodialysis module is configured to remove ammonium from the received spent culture media (i) and to output a reduced ammonium culture media (iii) and an ammonium containing concentrate (iv);
(d) a second electrodialysis module configured to receive:
(iii) the reduced ammonium culture media as a second diluate; and
(ii) an electrolyte solution, from the electrolyte solution reservoir (b) as a second concentrate,
wherein the second electrodialysis (d) is configured to remove lactate from the reduced ammonium culture media (iii) and to output a reduced ammonium and lactate culture media (P) and a lactate containing solution (Q);
(e) an ion exchange module configured to receive the ammonium containing concentrate (iv), to remove the ammonium and to output a reduced ammonium solution (R); and
(f) a third electrodialysis module configured to receive:
(v) the reduced ammonium solution (R) as diluate; and
(vi) the reduced ammonium and lactate culture media (P) as a third concentrate, wherein the third electrodialysis module (f) is configured to return the reduced ammonium solution (R) to the reduced ammonium and lactate culture media (P); and
to output:
an electrolyte enriched, ammonium reduced and lactate reduced culture media (L) to be returned to the culture media reservoir (a) thereby purifying the spent culture media, and
an electrolyte solution (M) to be returned to the electrolyte solution reservoir (b) for re-use as concentrate for the first dialysis module (c) and second dialysis module (d).

19. (canceled)

20. (canceled)

21. The recycling system of claim 18, wherein the reduced ammonium solution (R) output by the ion exchange module (e) comprises one or more vital materials that are returned to the culture media reservoir (a) through the third electrodialysis module (f).

22. (canceled)

23. (canceled)

24. The recycling system of claim 18, wherein the system is further configured to return to the ammonium reduced and lactate reduced culture media, a vital material that was removed during the recycling process.

25. The recycling system of claim 18, wherein the system is further configured to replace a depleted vital material in the culture media.

26. The recycling system of claim 25, wherein the vital material comprises a growth factor, an amino acid, a vitamin, a protein, an enzyme, a co-enzyme, a hormone, a sugar, a carbohydrate, a micronutrient, macronutrient, a mineral, an osmolarity agent, a pH maintenance agent, or any combination thereof.

27. The recycling system of claim 18, wherein the order of the at least one electrodialysis module and the at least one ion exchange module is variable.

28. The recycling system of claim 18, wherein the culture media is used for cultivated meat production, cultivated plant production, enzyme production, growth of artificial organs, alternative protein production, metabolites, production of pharmaceutically active components, or any combination thereof.

29. The recycling system of claim 18, wherein the culture media is used to cultivate cells comprising animal cells, plant cells, bacteria, yeast, fungi, microalgae, algae, or any combination thereof.

30. The recycling system of claim 18, wherein the culture media is used to cultivate tissue comprising animal tissue, plant tissue, fungal, algal tissue, or any combination thereof.

31. The recycling system of claim 18, wherein the culture media further comprises a growth factor, an amino acid, a vitamin, a protein, an enzyme, a co-enzyme, a hormone, a sugar, a carbohydrate, a micronutrient, macronutrient, a mineral, an osmolarity agent, a pH maintenance agent, or any combination thereof.

32. (canceled)

33. The recycling system of claim 18, wherein the configured to operate as a continuous process, semi-batch process, or a batch process.

34. The recycling system of claim 18, wherein an amount of electrolyte added to the reduced ammonium and lactate culture media is up to about 10% v/v.

35. The recycling system of claim 18, wherein the electrolyte is a salt solution comprising sodium, calcium, potassium, chloride, phosphate, carbonate, bicarbonate, magnesium, sulfonic acid, sulfate, nitrate, organic salts like acetate, citrate, formate, a derivative thereof, or any combination thereof.

36. The recycling system of claim 18, wherein the electrolyte comprises a NaCl solution.

37. The recycling system of claim 18, wherein the first electrodialysis module, the second electrodialysis module or the third electrodialysis module includes at least one 2-cell repeating unit including an Anion Exchange Membrane (AEM) and a Cation Exchange Membrane (CEM), wherein the number of repeat units is in the range between about 1-300 repeating units.

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. The recycling system of claim 18, wherein up to about 99% of the lactate is removed from the reduced ammonium culture media in the second electrodialysis module to output the reduced ammonium and lactate culture media.

54. (canceled)

55. (canceled)

56. (canceled)

57. (canceled)

58. (canceled)

59. (canceled)

60. (canceled)

61. The recycling system of claim 18, wherein up to about 99% of the ammonium is removed from the ammonium containing concentrate in the ion exchange module to output the reduced ammonium solution.

62. (canceled)

63. (canceled)

64. (canceled)

65. (canceled)

66. (canceled)

67. (canceled)

68. (canceled)

69. A method for recycling spent culture media by removing ammonium and lactate comprising:
(a) receiving:
spent culture media as a first diluate, from a culture media reservoir; and
an electrolyte solution, from an electrolyte solution reservoir, as a first concentrate;
to a first electrodialysis module configured for removing ammonium from the received spent culture media and outputting a reduced ammonium culture media and an ammonium containing concentrate;
the reduced ammonium culture media as a second diluate; and
an electrolyte solution, from an electrolyte solution reservoir, as a second concentrate;
to a second electrodialysis module configured for removing lactate from the reduced ammonium culture media and outputting a reduced ammonium and lactate culture media and a lactate containing solution;
(c) receiving the ammonium containing concentrate to an ion exchange module configured for removing the ammonium and for outputting a reduced ammonium solution;
(d) receiving:
the reduced ammonium solution as diluate; and
the reduced ammonium and lactate culture media as a third concentrate;
to a third electrodialysis module configured for returning the reduced ammonium solution to the reduced ammonium and lactate culture media and for outputting an electrolyte enriched ammonium reduced and lactate reduced culture media to be returned to the culture media reservoir thereby purifying the spent culture media, and
(e) returning an electrolyte solution to the electrolyte solution reservoir for re-using as concentrate for the first dialysis module (c) and second dialysis module (d).

70. A method for recycling spent culture media by removing ammonium and lactate comprising:
(a) receiving:
spent culture media as a first diluate, from a culture media reservoir; and
an electrolyte solution, from an electrolyte solution reservoir, as a first concentrate;
to a first electrodialysis module configured for removing ammonium from the received spent culture media and outputting a reduced ammonium culture media and an ammonium containing concentrate;
the reduced ammonium culture media as a second diluate; and
an electrolyte solution, from an electrolyte solution reservoir, as a second concentrate;
to a second electrodialysis module configured for removing lactate from the reduced ammonium culture media and outputting a reduced ammonium and lactate culture media and a lactate containing solution;
(c) receiving the ammonium containing concentrate to an ion exchange module configured for removing the ammonium and for outputting a reduced ammonium electrolyte solution, and
(d) returning the reduced ammonium electrolyte solution to the electrolyte solution reservoir for re-using as concentrate for the first dialysis module (b) and the second dialysis module (c);
(e) adding an electrolyte to the reduced ammonium and lactate culture media and outputting an electrolyte enriched ammonium reduced and lactate reduced culture media to be returned to the culture media reservoir thereby purifying the spent culture media.

71. The recycling system of claim 70, wherein the adding of electrolyte to the reduced ammonium and lactate culture media is in an amount up to 10% v/v.

72. (canceled)
